(19) United States
(12) Patent Application Publication
(10) Pub. No.: US 2005/0244482 A1
(43) Pub. Date: Nov. 3, 2005

(54) Complex face mask and methods of preparation thereof

(75) Inventors: Hsiao-Cheng Yen, TaoYuan County (TW); Jo-Yi Hsiao, TaoYuan County (TW)

Correspondence Address: BACON & THOMAS, PLLC, 625 SLATERS LANE, FOURTH FLOOR, ALEXANDRIA, VA, 22314, US

(73) Assignee: Life Spring Biotech. Co. Ltd., TaoYuan County (TW)

(21) Appl. No.: 10/982,929

(22) Filed: Nov. 8, 2004

Related U.S. Application Data
(60) Provisional application No. 60/566,937, filed on May 3, 2004.

(51) Int. Cl. A61K 9/70

(52) U.S. Cl. 424/443

(57) ABSTRACT

The present invention relates to a complex face mask comprising: a complex face mask, which comprises a face mask containing effective ingredients wherein the effective ingredients are attached to the face mask in freeze-drying form; and a skin care solution. Also disclosed is a method of producing a complex face mask, which comprises dousing a face mask cloth in a solution containing effective ingredients and then freeze-drying the face mask; and preparing a skin care solution. The effective ingredients attached to the freeze-drying face mask can fully show their effects so that the skin condition can be improved.

### BACKGROUND OF THE INVENTION

1. Field of the Invention

The present invention relates to a complex face mask and the production method thereof, in particular a complex face mask in which effective ingredients are preserved in the face mask by freeze-drying and the ingredients will not lose their effects because of time factor and the production method thereof.

2. Description of the Related Art

The face mask is a popular cosmetic used to take care of face skin. The significant effect of the face mask in moisturizing skin results from its strong water absorbing ability and hermetic effect. The strong water absorbing ability retains essences in the face mask, and the hermetic effect avoids water loss so that the substances in the face mask can fully penetrate into skin to obtain protective and nutritious effects. When a moisturized face mask is applied to the face skin, the substances contained therein slowly penetrate into the skin and the face mask will insulate the skin from air to avoid water loss from the face mask quickly.

Generally, the commercial face mask is produced by drenching with a prepared skin care solution containing effective ingredients and then directly packs for preserving. Alternatively, the face mask and the prepared skin care solution containing effective ingredients are individually packed wherein the skin care solution is poured to the face mask before applying to the face. However, some of the effective ingredients, such as Vitamin C, are so unstable that the known face masks containing those ingredients can not achieve the desired effect because the unstable ingredients are easily oxidized and lose effect when preserved in a hydrous condition.

It still has a need to develop a new face mask wherein the effective ingredients contained in face mask are active during storage period so that the face mask can exactly achieve the desired effect.

### SUMMARY OF THE INVENTION

One object of the invention is to provide a complex face mask which comprises a face mask containing effective ingredients wherein the effective ingredients are attached to the face mask in a freeze-drying form; and a skin care solution.

Another object of the invention is to provide a method of producing a complex face mask, which comprises dousing a face mask cloth in a solution containing effective ingredients and then freeze-drying the face mask; and preparing a skin care solution.

For accomplishing the object mentioned above, the present invention provides a complex face mask containing effective ingredients wherein the effective ingredients are attached to the face mask in a freeze-drying form; and a skin care solution.

According to the present invention, the effective ingredients of the face mask are effective in maintaining skin moisture, increasing the growth of derma tissue, preventing the skin from premature aging and wrinkles, improving skin elasticity, preventing UV damage, and reducing the brownish stains and pigmentation marks. The effective ingredients comprise, but not limited to, collagen, Vitamin C, hEGF (Human Epidermal Growth Factor), bioactive peptide, and other nourishing substances that are easily oxidized. For example, collagen is a protein polymer for making up extra cellular matrix, which plays a critical role in connecting tissues of animal. Vitamin C has effects in increasing the anabolism of collagen, preventing UV damage, inhibiting oxidation, and reducing the brownish stains and pigmentation marks. hEGF induces cells proliferation and regulates tissue differentiation. Bioactive peptide is a small molecule that can easily pass through epidermal tissue to stratum germinativum. The bioactive peptide can produce collagen by activating skin cells to firm skin, improve skin elasticity, and prevent damage from outside environment. The effective ingredients mentioned above are easily oxidized and loss activity.

According to the present invention, the effective ingredients are attached to the face mask in a freeze-drying form and the face mask is stored in a vacuumed shading package. Thus, the effective ingredients contained in the face mask can avoid being oxidized and well conserved until applying the face mask to skin to achieve desired effects.

According to the present invention, the skin care solution is prepared by adding skin care substances, which are different from effective ingredients contained in face mask and have softening and nourishing effects, to water and then fill into sterilized bottles for packing. The skin care solution comprises but not limited to Hyaluronic Acid, Liquoric, Witch Hazel Extract, and other nourishing materials, which are not oxidized while dissolved in water, and do not reduce activity.

The present invention provides a method of producing a complex face mask, which comprises dousing a face mask cloth in a solution containing effective ingredients and then freeze-drying the face mask; and preparing a skin care solution.

According to the present invention, the method of producing a complex face mask, wherein said dousing further comprises that the solution containing effective ingredients is prepared by adding a thickener into water and mixing; and adding the effective ingredients to the solution. The thickener is used in increasing the viscosity of solution so that the effective ingredients can easily attach to the face mask. The suitable thickener used in the invention comprises but not limited to natural or artificial products which will not react with the effective ingredients and not damage skin.

According to the present invention, the method of producing a complex face mask, wherein said face mask in freeze-drying form further comprises that the face mask cloth after freeze-drying is stored in a vacuumed shading package. The purpose of storing the face mask in a vacuum and no light condition is to avoid oxidation and maintain activity of the face mask before being used.

The effective ingredients of the present invention include collagen, Vitamin C, hEGF, bioactive peptide, and other nourishing materials which are easily oxidized and reduce activity.

According to the present invention, the manufacturing process of skin care solution is prepared by adding skin care substances, which are different from effective ingredients contained in face mask and have softening and nourishing effects, to water and then fill into a sterilized bottle for packing. The skin care solution comprises but not limited to Hyaluronic acid, Liquoric, Witch hazel extract, and other nourishing materials, which are not oxidized while dissolved in water, and do not reduce activity.

Method of using the complex face mask of the invention comprises adding the skin care solution to a freeze-dried face mask; at this moment, the effective ingredients attached on the face mark will be educed to the skin care solution; and then apply the face mask to the face for a period of time. The collagen of skin tissue fully absorbs water in a moist environment. The effective ingredients and skin care substances penetrate slowly to the deep skin. Meanwhile, because of capillary dilation and increasing of blood microcirculation, the effective ingredients and skin care substances are efficiently entered into skin cells. Therefore, the skin becomes whiter and more soft, elastic, and bright after applying the face mask of the invention.

To clearly express the objects, structure characters, and functions of the present invention, the detailed contents and preferred embodiment of the present invention are illustrated with reference to the attached figures.

### BRIEF DESCRIPTION OF THE DRAWINGS

Preferred embodiments of the invention will now be described with reference to the attached drawings in which:

FIG. 1 shows the complex face mask of the invention;

FIG. 2 shows the flow chart of producing freeze-drying face mask containing effective ingredients; and

FIG. 3 shows the process of preparing skin care solution.

### DETAILED DESCRIPTION OF PREFERRED EMBODIMENT OF THE INVENTION

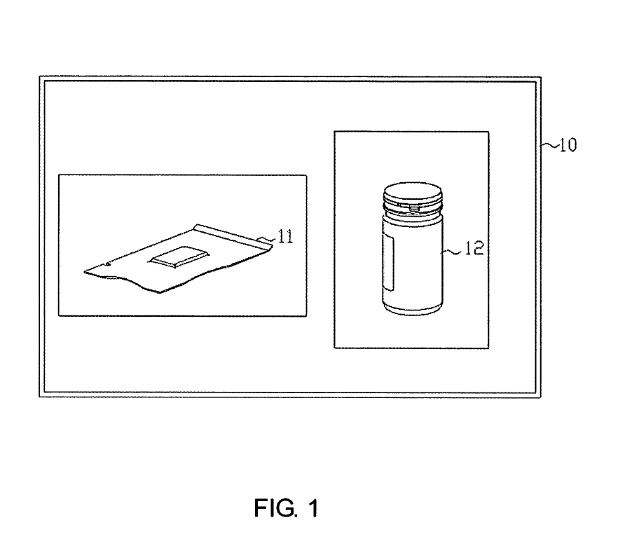

The method of producing a complex face mask, which comprises dousing a face mask cloth fully with a solution containing effective ingredients, and then proceeding freeze-drying; and preparing a skin care solution. The complex face mask 10 shown in FIG. 1 comprises a freeze-dried face mask 11 and a skin care solution 12.

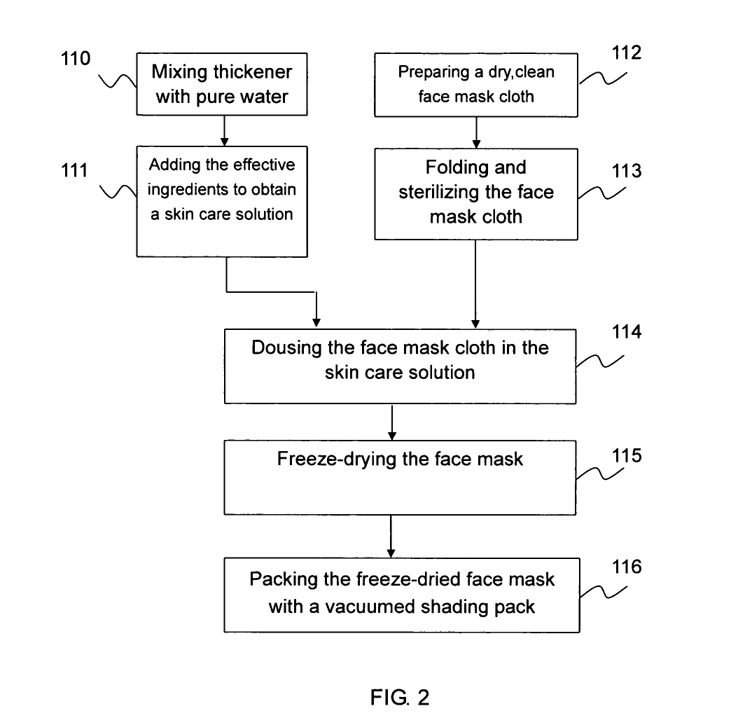

Said dousing a face mask cloth fully with a solution containing effective ingredients and then proceeding freeze-drying process, as shown in FIG. 2.
A dry, clean face mask cloth was prepared (step 112). Said face mask cloth was folded and carried out a γ ray sterilization (step 113) to obtain a sterilized face-mask cloth.
Thickener was added to pure water to obtain a thickener solution having a concentration of about 0.2% to 1.0% by weight and then mixed (step 110). The effective ingredients, such as collagen, Vitamin C, hEGF, bioactive peptide, etc., (step 111) were fully mixed with above-mentioned ingredients to obtain a skin care mask solution.
The sterilized face mask cloth was doused in the skin care solution (step 114) so that the effective ingredients were attached to the face mask to obtain the face mask cloth containing effective ingredients.
The face mask cloth containing effective ingredients was freeze-dried (step 115). In the freeze-drying process, the face mask cloth containing effective ingredients was first frozen in −40° C. for freezing water and effective ingredients of the face mask cloth. Then, the resultant face mask cloth was dried under a vacuum condition to remove the water contained in the face mask cloth. Consequently, the freeze-drying face mask was obtained.
The freeze-drying face mask was packed in a vacuumed shading package (step 116) to obtain the product of freeze-drying face mask 11.

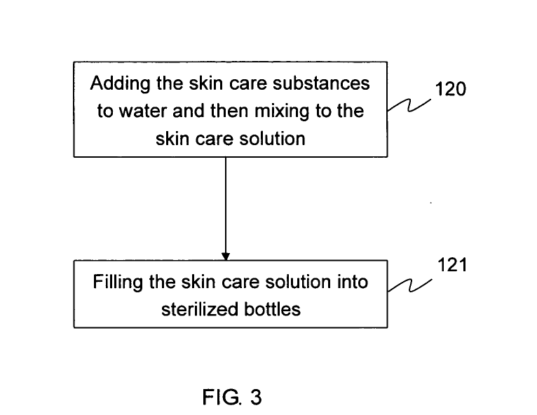

Said preparing a skin care solution, as shown in FIG. 3.
The skin care substances, such as Hyaluronic acid, Liquoric, Witch hazel extract, etc., were added to water and then mixed to the skin care solution (step 120).
The skin care solution was filled into sterilized bottles (step 121) to obtain the product of skin care solution 12.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope and spirit of the appended claims.

1. A complex face mask, which comprising:
a face mask containing effective ingredients wherein the effective ingredients are attached to the face mask in freeze-drying form; and
a skin care solution, which can apply to the face mask when being used.

2. The complex face mask according to claim 1, wherein said face mask is stored in a vacuumed shading package.

3. The complex face mask according to claim 1, wherein said effective ingredients include collagen, Vitamin C, hEGF, bioactive peptide, and other nourishing materials which are easily oxidized and reduce activity.

4. The complex face mask according to claim 1, wherein said skin care substances include Hyaluronic Acid, Liquoric, Witch Hazel Extract, and other nourishing materials which are not easily oxidized.

5. A method of producing a complex face mask, comprising:
dousing a face mask cloth in a solution containing effective ingredients and then freeze-drying said face mask; and
preparing a skin care solution.

6. The method of producing a complex face mask according to claim 5, wherein said freeze-drying face mask further comprising:
storing the freeze-dried face mask in a vacuumed shading package.

7. The method of producing a complex face mask according to claim 5, wherein said dousing a face mask cloth in a solution containing effective ingredients further comprising:
adding a thickener in pure water and then mixing to form a thickener solution; and
adding effective ingredients to said thickener solution and then mixing well into a skin care solution.

8. The method of producing a complex face mask according to claim 5, wherein said effective ingredients include collagen, Vitamin C, hEGF, bioactive peptide, and other nourishing materials which are easily oxidized and reduce activity.

9. The method of producing a complex face mask according to claim 5, wherein said skin care solution is prepared by dissolving the skin care substances in water.

10. The method of producing a complex face mask according to claim 5, wherein said skin care substances include Hyaluronic Acid, Liquoric, Witch Hazel Extract, and other nourishing materials which are not easily oxidized.
